(19) United States
(12) Patent Application Publication
(10) Pub. No.: US 2020/0104935 A1
(43) Pub. Date: Apr. 2, 2020

(54) SYSTEMS AND METHODS FOR WEALTH AND HEALTH PLANNING

(71) Applicant: FMR LLC, Boston, MA (US)

(72) Inventors: Suzanne Schmitt, Boston, MA (US); Sunil Madhani, Boston, MA (US)

(21) Appl. No.: 16/144,370

(22) Filed: Sep. 27, 2018

(51) Int. Cl. G06Q 40/06; G06Q 40/02; G16H 50/30

(52) U.S. Cl. CPC G06Q 40/06; G16H 50/30; G06Q 40/02

(57) ABSTRACT

A computerized method of generating a financial wellness score for retirement planning includes steps performed by a computing device including: receiving user information relating to demographic profile, financial health, physical health, psychosocial health, financial planning maturity and financial planning readiness of a user; categorizing the user into a career level classification based on demographic profile; identifying, based on the career level classification, one or more financial impact factors for the user; generating, based on the financial impact factors for the user and the user information relating to financial health, a future projected financial state; generating, based on the information relating to physical health and psychosocial health, a future projected health cost of the user; and calculating, based on the future projected financial state and the future projected health cost, a score indicating likelihood of achieving financial wellness in retirement.

### TECHNICAL FIELD

This application relates generally to systems, methods and apparatuses, including computer programs, for financial planning. More specifically, this application relates to generating a comprehensive retirement plan using financial and physical health data.

### BACKGROUND

According to statistics published by the Centers for Medicare and Medicaid Services, U.S. health care spending reached $3.3 trillion in 2016, or $10,348 per person. See https://www.cms.gov/Research-Statistics-Data-and-Systems/Statistics-Trends-and-Reports/NationalHealthExpendData/NationalHealthAccountsHistorical.html (page accessed Jul. 25, 2018). As a share of the nation's Gross Domestic Product, health spending accounted for 17.9 percent. However, the cost of healthcare is not distributed equally. By some estimates, approximately five percent of the population accounts for about 50% of the total medical costs. See, e.g., https://www.ncbi.nlm.nih.gov/books/NBK425792/ (page accessed Jul. 25, 2018). Thus, predicting individual healthcare costs with accuracy is critical to customizing workable retirement plans for investors of a wide range of wealth and risk profiles.

Current retirement plan calculations are based on limited numbers of variables, such as an investor's current assets, investment risk profile (e.g., conservative, medium, or aggressive risk taker), and expected retirement lifestyle. An investment plan is determined based on a formula that, for example, distributes current wealth in stocks, bonds and other assets in a pre-defined ratio that changes at definite intervals as the investor's age progresses. However, such plans neglect a host of relevant variables that vary from person to person and uniquely impact both projected healthcare costs over the course of an investor's lifetime and associated financial planning. What is needed is an approach to retirement planning that better accounts for the numerous variables relevant to wealth and health planning that fluctuate at the individual level, thereby creating a more holistic, safe and informed path to retirement.

### SUMMARY

Accordingly, the invention provides systems, methods and apparatuses to generate a personalized retirement plan based on a user's future projected financial state and the user's expected future healthcare costs. A “Wealth Health” or “Whealth” score is generated that accounts for many relevant but traditionally omitted variables, e.g., those relating to the user's financial, physical, and psycho-social well being, as well as prior financial planning and preparedness. Some exemplary variables include: significant health events; status as a caregiver; housing and/or relationship situation; mobility issues; social integration or isolation; susceptibility to fraud or abuse; and any preparation undertaken or guidance received in connection with negative life events (e.g., divorce, failure to launch, forced multi-generational house holding, debt, addiction issues, forced or premature retirement, aging issues, or death).

In this manner, Whealth planning “flips the script” on traditional financial planning—which is typically focused on financial matters only, such as accumulation of assets, savings, investments, and estate planning—by taking a 360° view of a customer's circumstances to develop a holistic financial plan into the future. Whealth planning also addresses common planning gaps, plan derailers and difficult family conversations, thus enabling customers to better protect their assets, preserve their lifestyles and maintain independence into older age. Because healthcare costs are uniquely variable from person to person, and are incurred to different degrees at different points in life for different people, healthcare costs can profoundly impact a customer's retirement planning. The invention's “WhealthCare Plan” generates a comprehensive and holistic lifetime retirement plan based on a connection between physical and financial health to develop a better-informed plan and to create a financial safety net.

As one exemplary setup, the invention can start by categorizing a customer between the ages of 20 to 80 (or older) into a “career-level classification category” (e.g., Early Career, Mid Career, Peak Earner, Pre Retiree, Early Retiree, Active Retiree, Mature Retiree). Then, for each career-level classification category at or above the customer's current age, the following information can be identified: key concerns; life events; blind spots; gaps and derailers; customer needs; and targeted opportunities. Next, key documents can be obtained for the user, such as bank account documents, medical directives, or legal documents, to verify and obtain further relevant information about the user. Then, information can be obtained about the customer via a survey in at least the following high level areas: financial health, physical health, social and psycho-social health. Based on the data collected, a score can be generated and a comprehensive retirement plan can be devised.

In one aspect, the invention features a computerized method of generating a financial wellness score for retirement planning. The computerized method includes receiving, by a computing device, user information relating to demographic profile, financial health, physical health, psychosocial health, financial planning maturity and financial planning readiness of a user. The computerized method also includes categorizing, by the computing device, the user into a career level classification based on demographic profile. The computerized method also includes identifying, by the computing device, based on the career level classification, one or more financial impact factors for the user. The computerized method also includes generating, by the computing device, based on the financial impact factors for the user and the user information relating to financial health, a future projected financial state. The computerized method also includes generating, by the computing device, based on the information relating to physical health and psychosocial health, a future projected health cost of the user. The computerized method also includes calculating, by the computing device, based on the future projected financial state and the future projected health cost, a score indicating likelihood of achieving financial wellness in retirement.

In some embodiments, the user information is received via a survey module including a set of survey questions administered to the user via the computing device. In some embodiments, the survey questions include questions on demographics, holistic health, financial wellbeing, physical wellbeing, psychosocial wellbeing and financial preparedness of the user. In some embodiments, identifying one or more financial impact factors for the user includes utilizing patterns extracted from a database of prior user information. In some embodiments, the computerized method includes using the financial wellness score to devise a personalized retirement plan. In some embodiments, the personalized retirement plan addresses at least one planning gap, possible plan derailer, family conversation, lifestyle preservation means, or plan to maintain independence into older age.

In some embodiments, the financial health information includes at least one of information on user budgeting, debt management, credit management, financial literacy, education planning or education saving. In some embodiments, the physical health information includes user information relating to at least one of user lifestyle conditions, chronic conditions, prescribed medications, body mass index, smoking habits, alcohol use, exercise habits, sleep habits, diet, safety, or driving habits. In some embodiments, the physical health information includes at least one of personal health, family health, or family health history. In some embodiments, the career level classifications include levels of “early career,” “mid-career,” “peak earner,” “pre-retiree,” “early retiree,” “active retiree,” and “mature retiree.” In some embodiments, the psychosocial health information includes information relating to at least one of common psychosocial conditions, results of user life choices, results of human relationships of the user, social connections of the user, stress management techniques of the user, identity management issues, or results of volunteerism by the user.

In some embodiments, the computerized method includes receiving at least one trigger document. In some embodiments, the act of completing a “trigger document” serves as a psychological prompt to take further action toward making a comprehensive financial plan. Action can mitigate inertia in planning and conversation and can help create momentum within a family to take the next best step in securing the family's future. In some embodiments, the trigger document is a beneficiary, medical directive, living will, medical order for life-sustaining treatment (MOLST), physician order for life-sustaining treatment (POLST), healthcare proxy, Health Insurance Portability and Accountability Act of 1996 (HIPAA) release form, power of attorney, guardian document, will, trust, letter of instruction, letter of intent, or family agreement. In some embodiments, the financial health information is based on indicia of at least one of a debt level, budgeting skill, credit management, financial literacy or education level of the user. In some embodiments, the financial wellness score is generated using a consumer diagnostic tool and a predictive model based on at least one of a meta-analysis of existing data sets and a health claims analysis.

In some embodiments, the computerized method further includes collecting, by the computing device, information on: (i) shake motion of the computing device collected via at least one of an accelerometer or a gyroscope of the computing device; (ii) color contrast settings or white point reduction settings of a display of the computing device; (iii) brightness level of the display of the computing device; (iv) usage of voice over or speak screen option; (v) usage of a zoom function of a display of the computing device; (vi) usage of an assistive touch function of the computing device; (vii) a number of times that an answer by the user changed for a given question; or (viii) a color filter setting of a display of the computing device.

In another aspect, the invention features a computing system for generating a retirement plan. The computing system includes a health cost model training module stored in memory of the computing system. The health cost model training module is configured to generate predictions of health costs based on external data. The computing system also includes a health trigger module stored in memory of the computing system and in electronic communication with the health cost training module. The health trigger module is configured to provide health information based on the predictions of health cost from the health cost training module. The computing system also includes a survey engine module stored in memory of the computing system and in electronic communication with the health trigger module. The survey engine module is configured to generate survey questions in a specified order based on the health information provided by the health trigger module. The computing system also includes a survey module stored in memory of the computing system and in electronic communication with the survey engine module. The survey module is configured to display the survey questions in the specified order for a user on a customer computing device in electronic communication with the computing system and to receive user answers to the survey questions via a user interface module in electronic communication with the computing system.

In some embodiments, the health trigger module is periodically updated and trained using updated user survey data comprising at least one of health issues or health cost issues. In some embodiments, the system includes a health care code cost database in electronic communication with the health cost model training module. In some embodiments, the system includes a customer health knowledge database in electronic communication with the health cost model training module. In some embodiments, the system includes a health savings account (HSA) customer withdrawal cost database in electronic communication with the health cost model training module. In some embodiments, the system includes a prescription medicine cost database in electronic communication with the health cost model training module. In some embodiments, the system includes a probabilistic health anomaly prediction engine in electronic communication with the health trigger module. The probabilistic health anomaly prediction engine is configured to generate trigger points on likely health issues of the customer.

In some embodiments, the system includes a customer settings table database in electronic communication with the health trigger module, the probabilistic health anomaly prediction engine configured to provide customer settings to the health trigger module. In some embodiments, the customer settings table database generates one or more clusters of settings based on common attributes of sensor settings recorded by the computing device as part of the survey module.

In another aspect, the invention includes a computerized method of training a probabilistic health anomaly prediction engine. The computerized method includes analyzing, by a computing device, existing national data sets including longitudinal study data sets. The computerized method also includes conducting, by the computing device, a net new utilization and consumption analysis. The computerized method also includes developing, by the computing device, a score-based recommendation and coaching plan.

In some embodiments, the invention can leverage proprietary data as well as data available from strategic partners. In some embodiments, the invention provides a Whealth management plan, e.g., the ability to pivot from an overarching score to stage-specific next best steps via a living plan. In some embodiments, the invention provides a Whealth management network, e.g., assesses the ecosystem of opportunities available to the customer and curates relationships, resources, and/or content that can support Whealth plans. In some embodiments, the invention accounts for event-based or change-based planning alterations, by which a customer can begin to understand “a-ha moments” driven by life events and their effects on the customer's overall Whealth plan (and adjust next steps accordingly).

In some embodiments, the invention accounts for inflation over time. In some embodiments, the invention accounts for varying co-pays or other cost changes in medical insurance. In some embodiments, the invention validates the truth of customer input or answers to survey questions by correlating with underlying legal documents. In some embodiments, the invention accounts for life events or changing events in a user's life and forecasts when medical events are likely to occur. In some embodiments, an expected variation of returns in a user's wealth and/or health plan can be calculated.

In some embodiments, the invention provides Whealth coaching, e.g., building on prior possibilities, offering comprehensive support, guidance, and content. In some embodiments, the invention can connect a customer with experts that provide end to end guidance, particularly around negative life events, unanticipated issues and common plan derailers. In some embodiments, the invention provides a family Whealth management plan, e.g., creates, models, or evolves individual plans and planning recommendations in the context of a broader family plan. For example, in the U.S., roughly 14.3% of the population acts in an unpaid caregiver capacity for another adult aged 50 or older. See, e.g., https://www.aarp.org/content/dam/aarp/ppi/2015/caregiving-in-the-united-states-2015-report-revised.pdf (page accessed Jul. 25, 2018). Among the affected population (including roughly 60% women) retirement savings can be affected, often in the range of hundreds of thousands of dollars or more (e.g., by having to leave the workforce prematurely). See, e.g., https://www.fidelity.com/viewpoints/personal-finance/caring-for-aging-parents (page accessed Jul. 25, 2018). Lack of planning (e.g., in the form of having proper long term care plans in place and having documents like healthcare proxies completed) and/or the consequences of chronic and lifestyle related health conditions have the potential to create a vicious cycle, whereby, for example, health events force people out of the workforce earlier than planned, causing them to incur additional expenses and require care that in turn forces family out of the workforce full time, reducing earnings and retirement savings, and causing them additional stress that results in health events for the caregiver, etc. The present invention can help mitigate this vicious cycle through early action, planning and preparedness.

### BRIEF DESCRIPTION OF THE DRAWINGS

The advantages of the invention described above, together with further advantages, may be better understood by referring to the following description taken in conjunction with the accompanying drawings. The drawings are not necessarily to scale; emphasis is instead generally placed upon illustrating the principles of the invention.

FIG. 1 is a schematic diagram of a computing system for generating a retirement plan, according to an illustrative embodiment of the invention.

FIG. 2 is a schematic diagram of a computerized method of generating a financial wellness score for retirement planning, according to an illustrative embodiment of the invention.

FIGS. 3A-3C are an illustrations of successive stages of a customer questionnaire eliciting relevant financial, physical, and psychosocial health information from a customer, according to an illustrative embodiment of the invention.

FIG. 4 is an illustration of a customer dashboard showing a “Wealth Health” or “Whealth” score summary, a future action plan summary, and a personal progress meter, according to an illustrative embodiment of the invention.

FIG. 5 is a schematic diagram of customer settings table database for a computing system for generating a retirement plan, according to an illustrative embodiment of the invention.

FIG. 6 is a schematic diagram of a computerized method of training a probabilistic health anomaly prediction engine, according to an illustrative embodiment of the invention.

### DETAILED DESCRIPTION

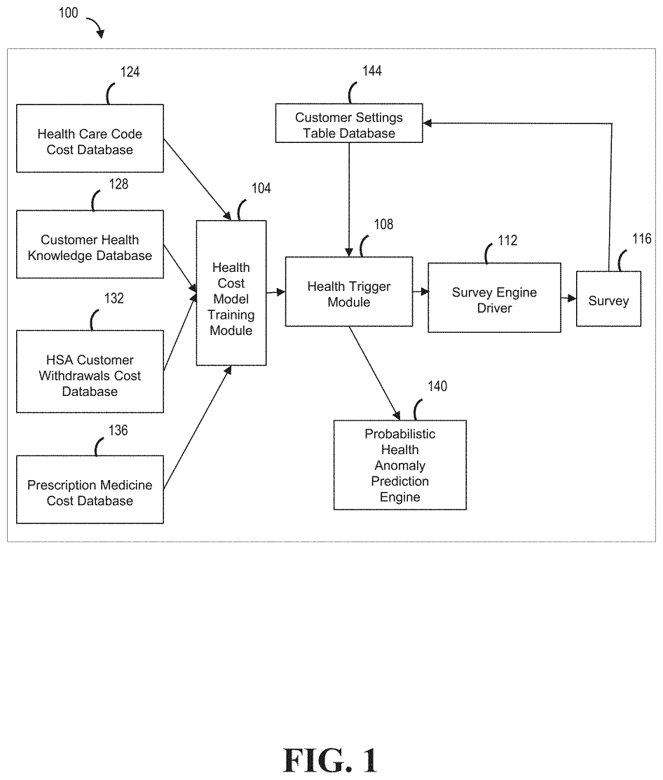

FIG. 1 is a schematic diagram of a computing system 100 for generating a retirement plan, according to an illustrative embodiment of the invention. The computing system 100 includes several modules that can include software (or hardware or combinations thereof) to execute the functions described herein. The computing system 100 includes a health cost model training module 104 stored in memory of the computing system 100. The health cost model training module 104 is configured to generate predictions of health costs based on external data, for example survey data, study data, or other systematically collected data. The computing system 100 also includes a health trigger module 108 stored in memory of the computing system 100 and in electronic communication with the health cost training module 104. The health trigger module 108 is configured to provide health information based on the predictions of health cost from the health cost training module 104. The health trigger module 108 can be periodically updated and trained using updated user survey data comprising at least one of health issues or health cost issues.

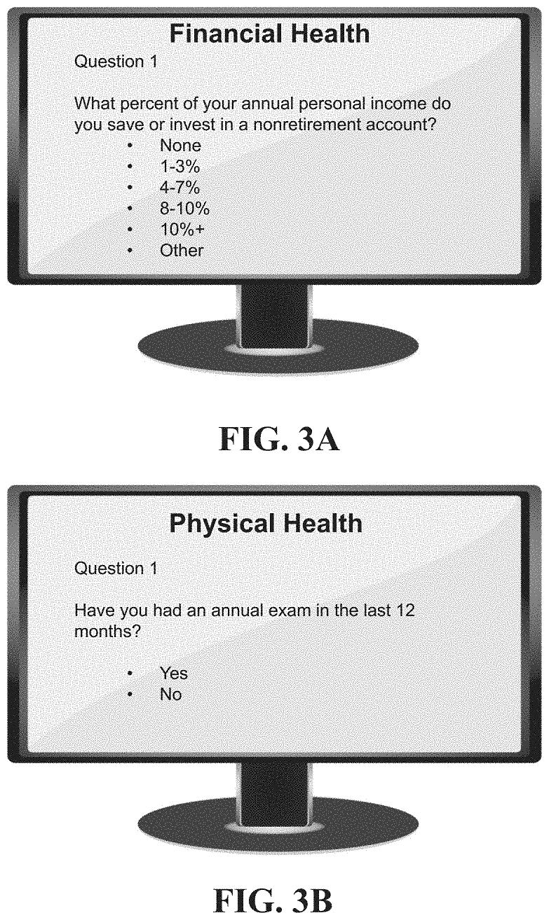

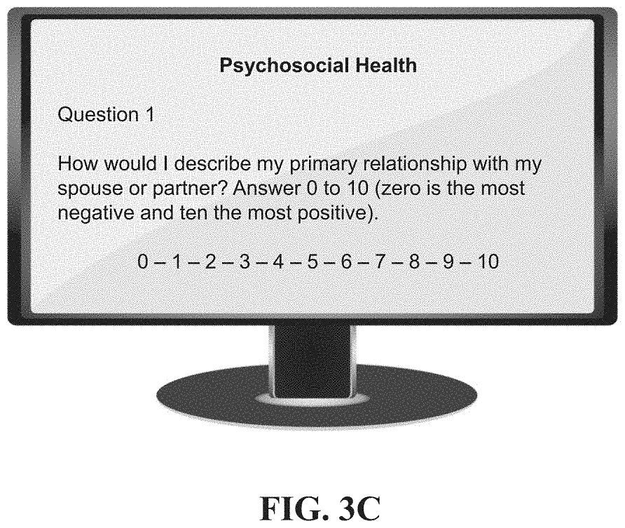

The computing system 100 also includes a survey engine module 112 stored in memory of the computing system 100 and in electronic communication with the health trigger module 108. The survey engine module 112 is configured to generate survey questions (e.g., in a specified order) based on the health information provided by the health trigger module 108. The computing system 100 also includes a survey module 116 stored in memory of the computing system 100 and in electronic communication with the survey engine module 112. The survey module 116 is configured to display the survey questions (e.g., in the specified order) for a user on a customer computing device 120 in electronic communication with the computing system 100 and to receive user answers to the survey questions via a user interface module (e.g., as shown below in FIGS. 3A-3C) in electronic communication with the computing system 100.

The computing system 100 can also include a health care code cost database 124 in electronic communication with the health cost model training module 104. The computing system 100 can also include a customer health knowledge database 128 in electronic communication with the health cost model training module 104. The computing system 100 can also include a health savings account (HSA) customer withdrawal cost database 132 in electronic communication with the health cost model training module 104. The computing system 100 can also include a prescription medicine cost database 136 in electronic communication with the health cost model training module 104.

The health care cost code database 124 can be provided, for example, via a health insurance service provider treatment code look-up. The customer health knowledge database 128 can include a historical tracking of an individual's health events as well as the individual's awareness of his or her current health, e.g., based on surveys. The health savings account (HSA) customer withdrawal cost database 132 can include a record of an individual's HSA withdrawal history. The prescription medicine cost database 136 can be an approximation of prescription costs based on a series of generics as well as proprietary drugs. Maintenance of the databases 124, 128, 132, 136 can be provided either through an API import for third party providers into the system or can be updated via individual activity (e.g., using real time data as well as project survey results).

The computing system 100 can also include a probabilistic health anomaly prediction engine (PHAPE) 140 in electronic communication with the health trigger module 108. The probabilistic health anomaly prediction engine 140 can be configured to generate trigger points on likely health issues of the customer. The computing system 100 can also include a customer settings table database 144 in electronic communication with the health trigger module 108. The probabilistic health anomaly prediction engine 140 can be configured to provide customer settings to the health trigger module 108. The customer settings table database 144 can generate one or more clusters of settings based on common attributes of sensor settings recorded by the computing system 100 as part of the survey module 116.

The health cost model training module 104 can be constantly updated and trained as more and more users take the survey and/or as certain health issues and health-related costs of past customers are recorded and analyzed. The health cost model training module 104 can also be updated based on external sources, e.g., the health care code cost database 124. The health trigger module 108 can run a routine periodically (e.g., every night) to detect any updates to the health cost model training module 104 or the customer settings table database 144. Desired settings can be fed into the health trigger module 108, which in turn can guide the survey engine driver 112 to direct the survey 116 to display certain sets of questions for the user and to specify a particular flow in which the questions are asked. Data from the customer settings table database 144 can be clustered based on common attributes and fed into the health trigger module 108. The health trigger module 108 can feed the customer settings table database 144 and knowledge acquired by the health cost model training module 104 to the probabilistic health anomaly prediction engine 140, which generates trigger points on likely health issues the customer might suffer or may suffer in the future. This approach may help, for example, in doing micro-level research on focused issues.

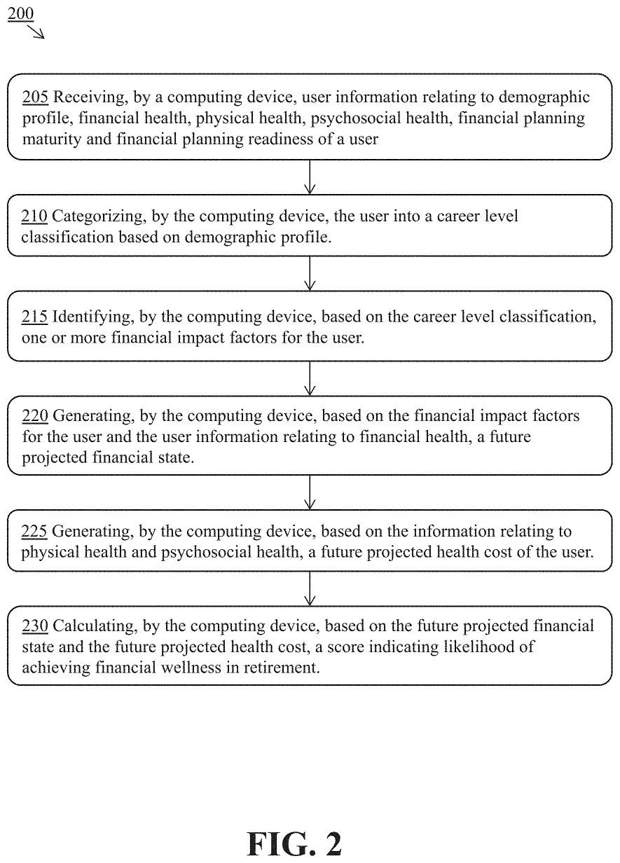

FIG. 2 is a schematic diagram of a computerized method 200 of generating a financial wellness score for retirement planning, according to an illustrative embodiment of the invention. In a first step 205, a computing device (e.g., the computing system 100 shown and described above in FIG. 1) receives user information relating to demographic profile, financial health, physical health, psychosocial health, financial planning maturity and/or financial preparedness or planning readiness of a user. For example, the financial health information can include at least one of information on user budgeting, debt management, credit management, financial literacy, education planning or education saving, and can be based on indicia of at least one of a debt level, budgeting skill, credit management, financial literacy or education level of the user. The physical health information can include user information relating to at least one of user lifestyle conditions, chronic conditions, prescribed medications, body mass index, smoking habits, alcohol use, exercise habits, sleep habits, diet, safety, or driving habits. The physical health information can also include at least one of personal health, family health, or family health history. The psychosocial health information can include information relating to at least one of common psychosocial conditions, results of user life choices, results of human relationships of the user, social connections of the user, stress management techniques of the user, identity management issues, or results of volunteerism by the user.

The information may be provided, for example, via a survey module (e.g., via the survey module 116 shown and described above in FIG. 1), which a user can use to take a survey having one or more questions in each of these segments or question areas. The responses to the survey questions can each be assigned a numerical value, which can then be weighted according to an algorithm and used to generate either a number (e.g., “H₁”) or a vector (e.g., “H₁, H₂, . . . H_{M}”).

In a second step 210, the computing device categorizes the user into a career level classification based on demographic profile. For example, the career level classifications can include levels of “early career,” “mid-career,” “peak earner,” “pre-retiree,” “early retiree,” “active retiree,” and “mature retiree.” The computing device can assign the responses from the demographic profile questions algebraic values, which can be inputted into an algorithm and used to generate either a number “C₁” or a vector “C₁, C₂, . . . C_{N}”. The career stage can be identified from [C₁, C₂, . . . C_{N}], where C₁ corresponds to “Early Career” and C_{N} corresponds to the last designated career stage, e.g., “Mature Retiree.” For example, whole numbers having a range 0-100 can be used for numerical values (and in some cases weighting and normalization operations may be needed to correct biases in later calculating the overall WHealth score). Certain algorithmic inputs, such as an individual's position in his or her career, may be naturally singular in nature. For example, a value of “0” can signify a pre-career individual (e.g., a student), and a value of 100 can signify a mature retiree. In some embodiments, a vector input (e.g., C₁, C₂, . . . C_{N}) can indicate growth of an individual's career over time. For example, an average progression may ascend from 10 to 20 to 30 over a 30-year time period, whereas a “fast-tracked” individual may ascend from 10 to 30 to 50 over the same time period.

In a third step 215, the computing device identifies, based on the career level classification, one or more financial impact factors for the user. These can be identified by utilizing patterns extracted from a database of prior user information. For example, they can be identified using a Whealth planning trajectory table (e.g., in a financial wellbeing part of financial health and life events section of the table). Life events from a financial perspective can include, for example, starting a career, getting married and/or divorced, purchasing a large item such as a house or a car, having children, funding education for dependents, obtaining a promotion, and entering retirement. Each of these events can be mapped onto a trajectory of Positives and Negatives. In some cases, individuals have more Negatives than Positives and accounts need to be balanced accordingly. In some cases, relevant information can be extracted from historical data on an individual via their financial statements.

In a fourth step 220, the computing device generates, based on the financial impact factors for the user and the user information relating to financial health, a future projected financial state. For example, the user information relating to “holistic health” (financial well-being, physical well-being, and psychosocial well-being) and financial preparedness can be assigned algebraic values, e.g., [H₁, H₂, . . . H_{M}], as discussed above. In some embodiments, these health indicators represent states of the individual at a certain moment in time and their historical health records, including family history, environment and aspects related to access to services (e.g. living in a city such as Boston versus in a remote rural area). In some embodiments, the individual can be surveyed regarding his or her well being and state of mind. In some embodiments, systematic input such as how an individual interacts with a smart device can also be used in this calculation. Based on user information in demographic section, the career stage is identified from [C₁, C₂, ... C_{N}], where C₁ corresponds to “Early Career” and C_{N} corresponds to “Mature retiree” stage.

In one exemplary embodiment, the financial state FS is calculated as follows. Let FSWM be the financial state weight matrix whose columns are represented by [H₁, H₂ . . . H_{M}] and whose rows are represented by [C₁, C₂, . . . C_{N}]. Elements of WM can be represented as W_{i,j} where j corresponds to a health value and i correspond to a career index stage. For example, W_{3,2} may represent a weight associated with a variable representing the health parameter H₂ for the career stage C₃. FSWM is then a matrix of m×n dimensions:

| | H₁ | H₂ | H₃ | H₄ | . . . | H_{M} |
|---|---|---|---|---|---|---|
| C₁ | W_{1,1} | W_{1,2} | W_{1,3} | W_{1,4} | . . . | W_{1,M} |
| C₂ | W_{2,1} | W_{2,2} | W_{2,3} | W_{2,4} | . . . | W_{2,M} |
| C₃ | W_{3,1} | W_{3,2} | W_{3,3} | W_{3,4} | . . . | W_{3,M} |
| . . . | . . . | . . . | . . . | . . . | . . . | . . . |
| C_{N} | W_{N,1} | W_{N,2} | W_{N,3} | . . . | . . . | W_{N,M} |

Then, the financial state FS is calculated to be a weighted average for a given career stage. For example, FS for C₂=H₁*W_{2,1}+H₂*W_{2,2}+ . . . +H_{M}*W_{2,M}. Then, the FS of a user for career stage Cᵢ=Σ(Wᵢ*H_{i,j}) for j=1 to m. Here for example, an individual at a mid-life mid-career stage point in time (C=50), could have their combined health & wealth index calculated to arrive at both an expected and actual index level. In some embodiments, the elements of FWSM are manually entered. In some embodiments, a hybrid update is utilized later (e.g., is entered manually and/or is generated by a “probabilistic health anomaly prediction engine,” e.g., as shown and described in FIG. 1.

In a fifth step 225, the computing device generates, based on the information relating to physical health and psychosocial health, a future projected health cost of the user. For this calculation, a Health Cost Weight Matrix (HCWM) can be used (similar to the FSWM shown and described above). HCWM can be a matrix of m×n with elements denoted by Z_{i,j}. If a user is in career stage Cᵢ, the projected health cost for career stage Cᵢ₊₁, Cᵢ₊₂, . . . C_{i+N} can be calculated as follows: Projected health Cost (PHC) for Career stage Cₓ=Σ(Zₓ*H_{x,j}) for j=1 to m. As above, in some embodiments, the elements of HCWM are manually entered. In some embodiments, a hybrid update is utilized later (e.g., is entered manually and/or is generated by a “probabilistic health anomaly prediction engine,” e.g., as shown and described in FIG. 1.

In a sixth step 230, the computing device calculates, based on the future projected financial state and the future projected health cost, a score indicating likelihood of achieving financial wellness in retirement. For a given career stage, the Financial State (FS) value can be computed as specified above, and the projected health care (PHC) can be computed for current and subsequent career stages. Then, the “Whealth” Score (WS) can be calculated as a mathematical operation on FS and PHC values. This score can correlate to how much a user should withdraw monthly and/or back-calculate for savings goals today.

Then, a financial wellness score can be used to devise a personalized retirement plan. The personalized retirement plan can address at least one planning gap, possible plan derailer, family conversation, lifestyle preservation means, or plan to maintain independence into older age. The financial wellness score can be generated using a consumer diagnostic tool and a predictive model based on at least one of a meta-analysis of existing data sets and a health claims analysis.

If there is a change in one or more of the above-discussed variables, a trigger can be sent to the Probabilistic Health Anomaly Prediction Engine (e.g., the PHAPE 140 shown and described above in FIG. 1) and relevant values can be re-computed. For example, if there is change in cost (e.g., a health care cost, prescription medicine cost or change in health funding), the PHAPE 140 can re-compute the FS (Financial state), Projected Health Cost (PHC) and WhealthScore (WS). If there is a change with respect to the originally computed score, the system can be updated accordingly. If there is an update to a user's health as reported directly by the user or while taking the survey, the PHAPE 140 can re-compute the Projected Health Cost (PHC) and WhealthScore (WS), and if there is a change with respect to the originally computed score, the system can be updated accordingly.

FIGS. 3A-3C are an illustrations of successive stages of a customer questionnaire eliciting relevant financial, physical, and psychosocial health information from a customer, according to an illustrative embodiment of the invention. For example, FIG. 3A shows the a question that a customer might first receive as part of the financial health segment of the questionnaire: “What percent of your annual personal income do you save or invest in a nonretirement account?” Similarly, FIG. 3B shows a question that a customer might first receive as part of the physical health segment: “Have you had an annual exam in the last 12 months?” Finally, FIG. 3C shows a question that a customer might first receive as part of the psychosocial health segment: “How would I describe my primary relationship with my spouse or partner? Answer 0 to 10 (zero is the most negative and ten the most positive).” Appendix A includes a more comprehensive list of exemplary questions that can be asked during each of these survey segments. In some embodiments, the customer questionnaire also has other segments, e.g., demographic questions and/or financial preparation and planning questions.

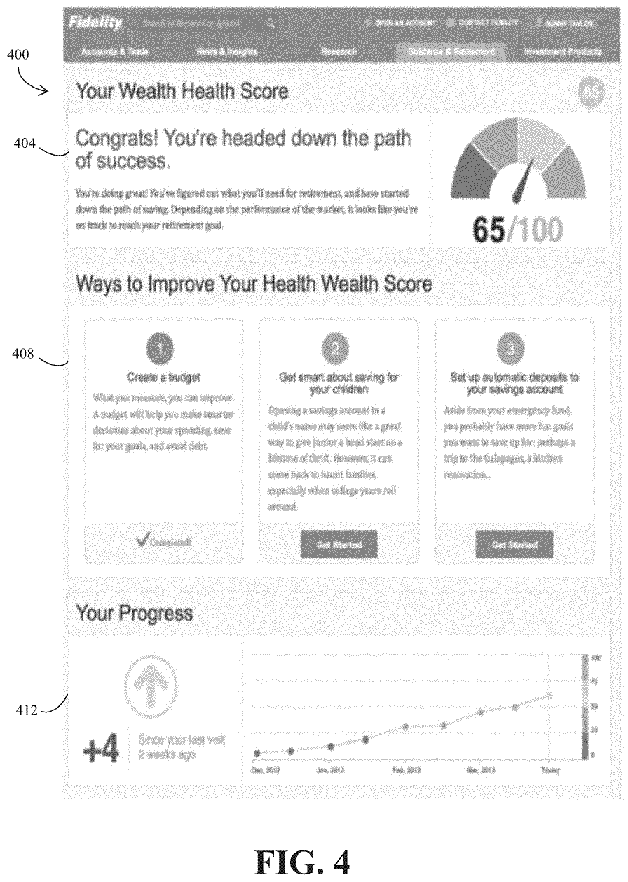

FIG. 4 is an illustration of a customer dashboard 400 showing a “Wealth Health” or “Whealth” score summary 404, a future action plan summary 408, and a personal progress meter 412, according to an illustrative embodiment of the invention. The “Whealth” score summary 404 can display a numerical score (e.g., out of 100) along with a brief blurb summarizing the customer's path to date and a more detailed synopsis of the same. For example, as depicted, the brief blur reads “Congrats! You're headed down the path of success” and the more detailed blurb reads “You're doing great! You've figured out what you'll need for retirement, and have started down the path of saving. Depending on the performance of the market, it looks like you're on track to reach your retirement goal.” The future action plan summary 408 can display enumerated “Ways to Improve Your Wealth Health Score,” e.g., as depicted (1) Create a budget; (2) Get smart about saving for your children; and (3) Set up automatic deposits to your savings account; and provide further details in a smaller print write-up below. The personal progress meter 412 can display a graph (e.g., a line graph) showing the progress of the Wealth Health score over time and/or a synopsis of progress made since the customer's last visit to the portal (e.g., “+4 Since your last visit 2 weeks ago,” as shown).

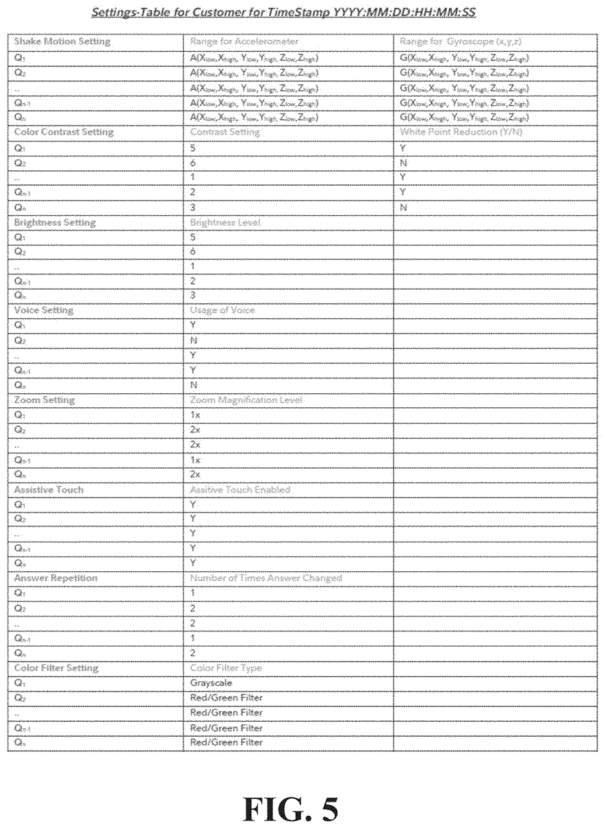

FIG. 5 is a schematic diagram of customer settings table database for a computing system for generating a retirement plan, according to an illustrative embodiment of the invention. In some embodiments, during a digital interaction with a customer (e.g., on an iPad), certain additional data can be captured that holds relevance to the calculations performed herein. For example, when a customer takes a WhealthCare Planning survey on an iPad, options can be provided to adjust certain settings to adapt to varying needs of the customer (which can vary, e.g., at different ages). The information can be stored in memory while the customer takes the survey. Storing setting preferences can help in reverting the survey display to the mode preferred by that customer. Based on a user's response while taking the survey on the computing device, recorded values from one or more sensors can be recorded in a customer settings table (e.g., the customer settings table database 144 shown and described above in FIG. 1), which can be used to correlate recorded data with one or more possible health issues. The customer settings table can be periodically updated (e.g., manually) based on experiential knowledge gained via research or iteratively better defined data sets gathered over time.

As implemented in the computerized method described above, that method can further include collecting, by the computing device, information on: (i) shake motion of the computing device collected via at least one of an accelerometer or a gyroscope of the computing device; (ii) color contrast settings or white point reduction settings of a display of the computing device; (iii) brightness level of the display of the computing device; (iv) usage of voice over or speak screen option; (v) usage of a zoom function of a display of the computing device; (vi) usage of an assistive touch function of the computing device; (vii) a number of times that an answer by the user changed for a given question; or (viii) a color filter setting of a display of the computing device. Regarding (i), unintentional rhythmic movements by the customer (e.g., tremors of hands) can be recorded. Tremors may be caused by problems with areas of the brain that control movements. Neurological problems can cause tremors, but they can also be caused by metabolic problems and toxins (such as alcohol) that affect the brain and nervous system. Shaking hands and tremor can also be a side effect of different medications. Regarding (ii) and (iii), any potential issues with eyesight, which may correlate with age, can be measured (e.g., measure the amount of transparency reduction and blur adjust for increased legibility). Regarding (iv), customer challenges relating to eyesight can be estimated, and the decibel level acceptable to a customer's ear can be gauged. Regarding (v), eyesight challenges of the customer can be estimated. Regarding (vi), unintentional rhythmic movements of the customer can be recorded. Regarding (vii), any issues with remembering or having an indecisive mind can be recorded. Regarding (viii), those who may have issues with color-blindness or have difficulty reading the text may be uncovered. For example, a mathematical model of the project degradation of physical attributes such as eyesight and/or hearing can be modeled against variables such as age, demographic, race, location. Taking this with device sensory information such as a user's ability to view devices of higher resolution, one can correlate both attributes to where the user would be expected to be at any moment in time.

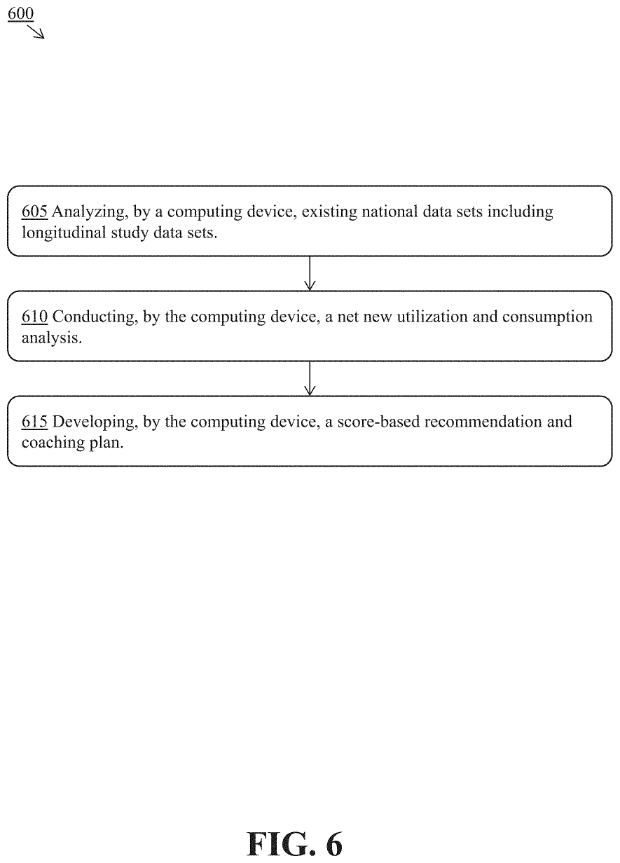

FIG. 6 is a schematic diagram of a computerized method 600 of training a probabilistic health anomaly prediction engine (PHAPE), according to an illustrative embodiment of the invention. In a first step 605, a computing device analyzes existing national data sets including longitudinal study data sets. For example, public information can be mapped to an array of anticipated performance results. Then, taking the input of the user, a correlation coefficient can be generated. In a second step 610, the computing device conducts a net new utilization and consumption analysis. Here the system can capture a user's interaction with his or her devices via biometric sensors (e.g., steps, heart rate, blood pressure, skin acid level), as well as their usage (e.g., higher contrast screens, font size, audio levels) and map to a projected index for a typical user of similar age, demographics, and/or location to calculate whether a user is on track or off track to a projected expected Whealth index. In a third step 615, the computing device develops a score-based recommendation and coaching plan. At this stage the computing device can have a projected anticipated health and wealth index to the individual's anticipated trajectory. If the computing device determines that the individual is on track, then there may be no need for change. If the computing device determines that the individual is off track, key areas for anticipated impact (e.g. lifestyle, fitness, wealth preparation for medical interventions etc.) can be proposed and tracked for progression against the individualized plan.

The above-described techniques can be implemented in digital and/or analog electronic circuitry, or in computer hardware, firmware, software, or in combinations of them. The implementation can be as a computer program product, i.e., a computer program tangibly embodied in a machine-readable storage device, for execution by, or to control the operation of, a data processing apparatus, e.g., a programmable processor, a computer, and/or multiple computers. The computer program can be deployed in a cloud computing environment (e.g., Amazon® AWS, Microsoft® Azure, IBM®). Method steps can be performed by one or more processors executing a computer program to perform functions of the invention by operating on input data and/or generating output data.

To provide for interaction with a user, the above described techniques can be implemented on a computing device in communication with a display device, e.g., a plasma or LCD (liquid crystal display) monitor or a mobile computing device display or screen for displaying information to the user and a keyboard and a pointing device, e.g., a mouse, a touchpad, or a motion sensor, by which the user can provide input to the computer (e.g., interact with a user interface element). Other kinds of devices can be used to provide for interaction with a user as well; for example, feedback provided to the user can be any form of sensory feedback, e.g., visual feedback, auditory feedback, or tactile feedback; and input from the user can be received in any form, including acoustic, speech, and/or tactile input.

The above-described techniques can be implemented in a distributed computing system that includes a back-end component. The back-end component can, for example, be a data server, a middleware component, and/or an application server. The above described techniques can be implemented in a distributed computing system that includes a front-end component. The front-end component can, for example, be a client computer having a graphical user interface, a Web browser through which a user can interact with an example implementation, and/or other graphical user interfaces for a transmitting device. The above described techniques can be implemented in a distributed computing system that includes any combination of such back-end, middleware, or front-end components.

The components of the computing system can be interconnected by transmission medium, which can include any form or medium of digital or analog data communication (e.g., a communication network). Transmission medium can include one or more packet-based networks and/or one or more circuit-based networks in any configuration. Packet-based networks can include, for example, the Internet, a carrier internet protocol (IP) network (e.g., local area network (LAN), wide area network (WAN), campus area network (CAN), metropolitan area network (MAN), home area network (HAN)), a private IP network, an IP private branch exchange (IPBX), a wireless network (e.g., radio access network (RAN), Bluetooth, near field communications (NFC) network, Wi-Fi, WiMAX, general packet radio service (GPRS) network, HiperLAN), and/or other packet-based networks. Circuit-based networks can include, for example, the public switched telephone network (PSTN), a legacy private branch exchange (PBX), a wireless network (e.g., RAN, code-division multiple access (CDMA) network, time division multiple access (TDMA) network, global system for mobile communications (GSM) network), and/or other circuit-based networks.

Information transfer over transmission medium can be based on one or more communication protocols. Communication protocols can include, for example, Ethernet protocol, Internet Protocol (IP), Voice over IP (VOIP), a Peer-to-Peer (P2P) protocol, Hypertext Transfer Protocol (HTTP), Session Initiation Protocol (SIP), H.323, Media Gateway Control Protocol (MGCP), Signaling System #7 (SS7), a Global System for Mobile Communications (GSM) protocol, a Push-to-Talk (PTT) protocol, a PTT over Cellular (POC) protocol, Universal Mobile Telecommunications System (UMTS), 3GPP Long Term Evolution (LTE) and/or other communication protocols.

Devices of the computing system can include, for example, a computer, a computer with a browser device, a telephone, an IP phone, a mobile computing device (e.g., cellular phone, personal digital assistant (PDA) device, smart phone, tablet, laptop computer, electronic mail device), and/or other communication devices. The browser device includes, for example, a computer (e.g., desktop computer and/or laptop computer) with a World Wide Web browser (e.g., Chrome™ from Google, Inc., Microsoft® Internet Explorer® available from Microsoft Corporation, and/or Mozilla® Firefox available from Mozilla Corporation). Mobile computing device include, for example, a Blackberry® from Research in Motion, an iPhone® from Apple Corporation, and/or an Android™-based device. IP phones include, for example, a Cisco® Unified IP Phone 7985G and/or a Cisco® Unified Wireless Phone 7920 available from Cisco Systems, Inc.

It should also be understood that various aspects and embodiments of the technology can be combined in various ways. Based on the teachings of this specification, a person of ordinary skill in the art can readily determine how to combine these various embodiments. In addition, modifications may occur to those skilled in the art upon reading the specification.

1. A computerized method of generating a financial wellness score and personalized plan for retirement planning, the computerized method comprising:
receiving, by a computing device, via a computing survey module of the computing device, user information including computing input data reflecting a set of answers to user survey questions administered to the user via the computing device, the survey questions relating to demographic profile, financial health, physical health, psychosocial health, financial planning maturity and financial planning readiness of a user;
assigning, by the computing device, a numerical value to each user survey question answer reflected in the computing input data;
calculating, by the computing device, a first number or vector based on a weighted sum of each numerical value multiplied by a weighting factor;
categorizing, by the computing device, the user into a career level classification based on demographic profile, the career level classification represented by a second number or vector;
identifying, by the computing device, based on the career level classification, one or more financial impact factors for the user according to a pattern based on a database of prior user information trained by a computing health cost model training module of the computing device;
generating, by the computing device, based on the financial impact factors for the user and the user information relating to financial health, a future projected financial state using a probabilistic health anomaly prediction engine (PHAPE) of the computing device utilizing a financial state weight matrix;
generating, by the computing device, based on the information relating to physical health and psychosocial health, a future projected health cost of the user using the PHAPE utilizing a health cost weight matrix;
calculating, by the computing device, based on the future projected financial state and the future projected health cost, a score indicating likelihood of achieving financial wellness in retirement, the score generated using a predictive model based on at least one of a meta-analysis of existing data sets and a health claims analysis; and
generating, by the computing device, based on the score, a personalized retirement plan including at least one recommendation for improving the score and addressing at least one planning gap, possible plan derailer, family conversation, lifestyle preservation means, or plan to maintain independence into older age.

2. (canceled)

3. (canceled)

4. (canceled)

5. (canceled)

6. (canceled)

7. The method of claim 1 wherein the financial health information includes at least one of information on user budgeting, debt management, credit management, financial literacy, education planning or education saving.

8. The method of claim 1 wherein the physical health information includes user information relating to at least one of user lifestyle conditions, chronic conditions, prescribed medications, body mass index, smoking habits, alcohol use, exercise habits, sleep habits, diet, safety, or driving habits.

9. The method of claim 1 wherein the physical health information includes at least one of personal health, family health, or family health history.

10. The method of claim 1 wherein the career level classifications include levels of “early career,” “mid-career,” “peak earner,” “pre-retiree,” “early retiree,” “active retiree,” and “mature retiree.”

11. The method of claim 1 wherein the psychosocial health information includes information relating to at least one of common psychosocial conditions, results of user life choices, results of human relationships of the user, social connections of the user, stress management techniques of the user, identity management issues, or results of volunteerism by the user.

12. The method of claim 1 further including receiving at least one trigger document.

13. The method of claim 12 wherein the trigger document is a beneficiary, medical directive, living will, medical order for life-sustaining treatment (MOLST), physician order for life-sustaining treatment (POLST), healthcare proxy, Health Insurance Portability and Accountability Act of 1996 (HIPAA) release form, power of attorney, guardian document, will, trust, letter of instruction, letter of intent, or family agreement.

14. The method of claim 1 wherein the financial health information is based on indicia of at least one of a debt level, budgeting skill, credit management, financial literacy or education level of the user.

15. (canceled)

16. The method of claim 1 further including collecting, by the computing device, information on: (i) shake motion of the computing device collected via at least one of an accelerometer or a gyroscope of the computing device; (ii) color contrast settings or white point reduction settings of a display of the computing device; (iii) brightness level of the display of the computing device; (iv) usage of voice over or speak screen option; (v) usage of a zoom function of a display of the computing device; (vi) usage of an assistive touch function of the computing device; (vii) a number of times that an answer by the user changed for a given question; or (viii) a color filter setting of a display of the computing device.

17. A computing system for generating a retirement plan, the computing system comprising:
a health cost model training module stored in memory of the computing system, the health cost model training module configured to generate predictions of health costs based on external data;
a health trigger module stored in memory of the computing system and in electronic communication with the health cost training module, the health trigger module configured to provide health information based on the predictions of health cost from the health cost training module;
a survey engine module stored in memory of the computing system and in electronic communication with the health trigger module, the survey engine module configured to generate survey questions in a specified order based on the health information provided by the health trigger module; and
a survey module stored in memory of the computing system and in electronic communication with the survey engine module, the survey module configured to display the survey questions in the specified order for a user on a customer computing device in electronic communication with the computing system and to receive user answers to the survey questions via a user interface module in electronic communication with the computing system.

18. The system of claim 17 wherein the health trigger module is periodically updated and trained using updated user survey data comprising at least one of health issues or health cost issues.

19. The system of claim 17 further including a health care code cost database in electronic communication with the health cost model training module.

20. The system of claim 17 further including a customer health knowledge database in electronic communication with the health cost model training module.

21. The system of claim 17 further including a health savings account (HSA) customer withdrawal cost database in electronic communication with the health cost model training module.

22. The system of claim 17 further including a prescription medicine cost database in electronic communication with the health cost model training module.

23. The system of claim 17 further including a probabilistic health anomaly prediction engine in electronic communication with the health trigger module, the probabilistic health anomaly prediction engine configured to generate trigger points on likely health issues of the customer.

24. The system of claim 17 further including a customer settings table database in electronic communication with the health trigger module, the probabilistic health anomaly prediction engine configured to provide customer settings to the health trigger module.

25. The system of claim 24 wherein the customer settings table database generates one or more clusters of settings based on common attributes of sensor settings recorded by the computing device as part of the survey module.

26. A computerized method of training a probabilistic health anomaly prediction engine, the computerized method comprising:
analyzing, by a computing device, existing national data sets including longitudinal study data sets;
conducting, by the computing device, a net new utilization and consumption analysis; and
developing, by the computing device, a score-based recommendation and coaching plan.

27. The method of claim 1 further comprising:
receiving, by the PHAPE, a trigger reflecting a change in the user information;
re-generating, by the PHAPE, the future projected financial state and the future projected health cost;
re-calculating, by the computing device, the score using the predictive model; and
re-generating, by the computing device, based on the re-computed score, an updated personalized retirement plan.

28. The method of claim 1 wherein the future projected health cost is based on at least one of a health care cost or a prescription medicine cost, the method further comprising:
receiving, by the PHAPE, a trigger reflecting a change in the health care cost or the prescription medicine cost;
re-generating, by the PHAPE, the future projected financial state and the future projected health cost;
re-calculating, by the computing device, the score using the predictive model; and
re-generating, by the computing device, based on the re-computed score, an updated personalized retirement plan.
